(19) United States
(12) Patent Application Publication
(10) Pub. No.: US 2009/0087390 A1
(43) Pub. Date: Apr. 2, 2009

(54) Fluidized slurry of water soluble and or water-swellable polymer and mixture thereof (FPS) for use in dentifrice and household applications

(76) Inventor: Jashawant J. Modi, Hockessin, DE (US)

Correspondence Address: Hercules Incorporated;Hercules Plaza, 1313 North Market Street, Wilmington, DE, 19894-0001, US

(21) Appl. No.: 12/284,910

(22) Filed: Sep. 26, 2008

Related U.S. Application Data
(60) Provisional application No. 60/995,519, filed on Sep. 27, 2007.

(51) Int. Cl. A61K 8/18; C11D 3/37; A61K 8/73

(52) U.S. Cl. 424/49; 510/397

(57) ABSTRACT

An oral care or household cleaner system contains a fluid polymer suspension (FPS) thickening agent. The FPS thickening agents comprises a water soluble/water swellable polymer, a polyol, and a hydrated thickening silica. The water soluble/water swellable polymer is selected from the group consisting of acrylic acid polymer, salts of acrylic acid polymer, acrylate, acrylamide polymer, polyvinyl pyrolidene, polyvinyl alcohol, clay and polysaccharide, excluding CMC. The functional systems may be formulations of oral care, for example as a dentifrice or as a household cleaner. A method of preparing the oral care or household cleaner system is also provided by adding a sufficient amount of a FPS that is compatible with the functional system to the system to thicken the functional system.

### FIELD OF THE INVENTION

This invention relates to dentifrice and household compositions containing fluidized polymer suspensions of a water soluble/water swellable polymer, a polyol, and hydrated thickening silica, wherein the water soluble/water swellable polymer is selected from the group consisting of polysaccharide, excluding CMC, acrylic acid polymer, salts of acrylic acid polymer, acrylate, acrylamide polymer polyvinyl pyrolidene, polyvinyl alcohol, and clay.

### BACKGROUND OF INVENTION

Dentrifrice formulations generally contain dentally acceptable abrasive, humectant, water, and water-soluble polymer which serve as a thickener and binder for the ingredients. A variety of other ingredients such as flavors, sweeteners, whitening agent, antimicrobial agent, tarter control agent salts, detergents, preservatives and fluoride are also utilized at low levels. Glycerin and sorbitol are the most commonly used humectants for toothpaste, and depending on the characteristics desired in the product, polyethylene glycol or polypropylene glycol may be incorporated as well. Dentifrice is also known as toothpaste. Two types of toothpaste are widely produced: 1) cream or opaque; and 2) transparent or translucent gel.

Household/institutional cleaners formulations generally contain an abrasive, acid, alkali, chealating agent, preservative, anti-bacteria agent, fragrance, water, and water-soluble polymer which serve as a thickener. A variety of other ingredients such as alcohol, salts, and detergents used.

In toothpaste the most commonly used thickeners or binders are Carboxymethyl cellulose (CMC) and hydroxyethyl cellulose (HEC). Carrageenan, xanthan, silica and polyacrylates are also used, but much less widely.

In household and institutional applications, the most commonly used thickeners for are xanthan, hydroxyethyl cellulose, and carbomer.

In the manufacturing process for toothpastes, incorporation of a dry water-soluble binder polymer into the toothpaste composition often presents difficulties because of the tendency for lump formation when the dry polymers are added to and dispersed in aqueous systems. Attempts to avoid this problem have resulted in several typical procedures for toothpaste manufacture. In the first typical procedure, the binder polymer is predispersed in a non-solvent (glycerin is preferred to wet out the binder polymer), and then a sorbitol/water mixture is added with vigorous agitation. If there is no (or only a low level of) glycerin or other water-miscible organic liquid in the toothpaste formulation, then all the available sorbitol is used with as much free water as possible with vigorous agitation. In the second typical procedure, the binder polymer is dry blended with other dry materials in the toothpaste formulation, e.g. abrasive, and then the resulting blend is dispersed in the liquid ingredients with strong agitation. To obtain a good dispersion, the binder polymer should be less than about 20% of the total dry blend. In this method, it is important to avoid blending the binder polymer with materials that compete for water, e.g., salts, sweeteners, preservatives, chealating agents.

In the manufacturing process for household composition such as hard surface cleaners, toilet blow cleaners, oven cleaners, incorporation of the dry water-soluble binder polymer into the household composition often presents difficulties because of the tendency for lump formation when the dry polymers are added to and dispersed in aqueous systems. These systems often do not contain a non-solvent to slurry the binder polymer in to non-solvent. Other formulation ingredients such as acids, alkali, abrasives, fragrance, preservatives, chealating agents, may be combined with the binder polymer to form the household composition.

These dispersion techniques are very time consuming for the manufacturer. Consequently, there is a need in the industry for methods of incorporating water-soluble binder polymers which lead to lump-free products, rapid viscosity development and reduced batch preparation time, and which allow for convenient handling of the binder polymer.

U.S. Pat. No. 3,574,824 discloses an anhydrous toothpaste base having the following ingredients: an oil, such as mineral oil or vegetable oil, with a viscosity of 100 to 300 cps; a combination of polyethylene glycols having a viscosity of 2,200 to 3,400 cps with molecular weights of 550 to 6,000; a non-toxic and non-ionic emulsifier; at least one binding agent; and a compound having a negative heat of hydration. Binding agents disclosed are polyvinylpyrrolidone, colloidal magnesium aluminum silicate, gum acacia, sodium alginate, extract of Irish moss, carboxymethyl cellulose and methyl cellulose. Hexahydric alcohols, in particular mannitol and inositol, are disclosed as compounds having a negative heat of hydration.

U.S. Pat. No. 5,192,529 teaches a dentifrice composition that contains a polyol humectant, abrasive, and a thickener system consisting of low viscosity sodium carboxymethyl cellulose and low viscosity hydroxyethyl cellulose.

U.K. Patent No. 1,363,182 discloses a transparent or translucent gel toothpaste composition comprising polyethylene glycol having molecular weight below 700, and hydrated silica gel polishing agent having a specific surface area below 600 m²/g. The composition may be thickened with carboxymethyl cellulose.

U.S. Patent No. 5,932,193, incorporated herein by reference in its entirety, discloses the use of a PEG based suspension for use in toothpaste formulations. All additives in the slurry composition are used in oral care applications. The preferred composition of the suspension was specified as (a) 40-60% of polyethylene glycol, (b) 0.5-2.0% of amorphous fumed silica, and (c) 40-60% of CMC.

Since it is well known in the prior art that the use of dry polysaccharides and prior art dispersion techniques are time consuming, inefficient, and not always compatible with functional systems in which they are added, there remains a need in the industry for incorporating water-soluble binder polymers into functional systems which provide lump-free products, rapid viscosity development, reduced batch preparation time, compatibility, and convenient handling of the binder polymer.

None of the above mentioned prior art meets all of the requirements of the needs of the industries.

### SUMMARY OF THE INVENTION

The present invention is directed to a composition an oral care and household cleaner system comprising a fluid polymer suspension (FPS) system. The FPS system comprises a water soluble/water swellable thickening polymer, a polyol, and a hydrated thickening silica. The water soluble/water swellable polymer is selected from the group consisting of, acrylic acid polymer, salts of acrylic acid polymer, acrylate, acrylamide polymer polyvinyl pyrolidene, polyvinyl alcohol, clay, and polysaccharide, excluding CMC.

The oral care and household cleaner system of the present invention can be any system comprising a water soluble/water swellable polymer, a polyol, and a hydrated thickening silica for use in oral care systems, such as a dentifrice composition and household cleaner system such as toilet bowl cleaners and tile cleaners.

The present invention is also related to a method of preparing an oral care household cleaner system comprising adding a sufficient amount of a fluid polymer suspension (FPS) that is compatible with the oral care and household cleaner system to thicken the oral care or household cleaner system. The FPS includes a water soluble/water swellable polymer, a polyol, and a hydrated thickening silica.

### BRIEF DESCRIPTION OF FIGURES

FIG. 1 depicts a graph of the hydration rate of xanthan in powder form versus xanthan in a fluidized polymer suspension.

### DETAILED DESCRIPTION OF THE INVENTION

It was surprisingly found that the present invention expands the scope of applications for polyol based suspensions beyond the uses disclosed in the prior art. It has been found that oral care systems, when using an FPS of a water soluble/water swellable polymer, polyol, and hydrated thickening silica, will provide a product that has comparable or better rheology and viscosity properties as compared to when using similar prior art thickening agents. The FPS not only eliminates problems of lumping and slow dispersion as compared to when using dry polymer, but also provides a more hazard free environment, more efficient system, a more tailored system for a particular task, and, in certain instances, multiple tasks in the system.

In addition, it was found that the toothpaste made with the product of this invention had improved gloss (sheen, smoothness) than one made with addition of a dry thickening agent.

In accordance with the present invention, the fluidized polymer suspension comprises a suspension of a water soluble/water swellable polymer, preferably a polysaccharide, but excluding CMC, in a polyol, preferably a polyethylene glycol. In the suspensions, the polysaccharide is in the form of a finely divided solid. Preferably, the particle size distribution of the polysaccharide is such that about 80% of the particles have a size less than about 75 μm.

The polyol of use in the FPS of present invention is selected from the group consisting of, glycerol, polyethylene glycol, propylene glycol, and mixtures thereof.

The upper limit of the amount of the polyol based on the total weight of the FPS is about 90 wt %, preferably about 80 wt %, more preferably about 70 wt %. The lower limit of the amount of polyol based on the total weight of the FPS is about 40 wt %, preferably about 50 wt %, more preferably about 60 wt %.

Although any water miscible, polyol may be used in the fluidized polymer suspension of the present invention, the preferred polyols are PEGs with mean average molecular weight (Mw) of less than about 1,000. More preferred are the PEGs with Mw of less than about 700, and most preferred are those with Mw in the range of about 200 to about 400.

The ingredients, contained in the composition of PEG based FPS of the present invention, are approved by the FDA for use in food contact. PEG 300 and 400 are approved under 21 CFR 176.170 and 176.180 and Aerosil® 200 silica (available from Evonik Industries), complies with FDA regulation under 21 CFR 176.200 and 176.210 for use as indirect food additive. Furthermore, both PEG 300 and 400 and Aerosil® silica are used in oral care applications in contact with mucous membranes.

In accordance with the present invention, normally, the maximum amount of the PEG used in the fluidized polymer suspension is about 90% by weight based on the total weight of the FPS. Preferably, the maximum (or upper limit) amount of the PEG is about 80 wt % and more preferably about 70 wt %. The normal minimum (or lower limit) amount of the PEG is about 40 wt %, preferably about 50 wt %, and more preferably about 60 wt %.

Hydrated thickening silica is incorporated in the FPS of the present invention to serve as a suspending agent for the dispersed water-soluble polymer. Hydrated thickening silicas are synthetic silicas including fumed silicas, hydrophobic modified fumed silica, hydrophilic modified fumed silica, amorphous precipitated silicas, and gel silicas, including modified silica including mixtures thereof. The fumed silica has a small particle size (approx. 10 μm) and large surface area (200 to 300 m²/g), making fumed silica very effective for thickening, thixotropy, and as a suspending vehicle at low concentrations.

The upper limit (or maximum) amount of hydrated thickening silica that is used in the FPS of the present invention is about 5.0% by weight based the total weight of the FPS, preferably about 3.0% by weight and more preferably about 2.0 wt %. The lower limit amount of the hydrated thickening silica is about 0.1 wt %, preferably about 0.5 wt %.

In accordance with the present invention, polysaccharide is at least one member selected from the group consisting of cellulose ethers (excluding CMC alone), guar, guar derivatives, locust bean gum, psyllium, gum arabic, gum ghatti, gum karaya, gum tragacanth, carrageenan, konjac, agar, alginates, xanthan, scleroglucan, dextran, pectin, starch, starch derivatives, chitin, chitosan, and processed euchemia seaweed (PES), derivatives and mixture thereof.

The cellulose ether of use in the present invention may be selected from the group consisting of hydroxyethyl cellulose (HEC), hydroxypropyl cellulose (HPC), water soluble ethylhydroxyethyl cellulose (EHEC), carboxymethylhydroxyethyl cellulose (CMHEC), hydroxypropylhydroxyethyl cellulose (HPHEC), methyl cellulose (MC), methylhydroxypropyl cellulose (MHPC) (when used in combination with other polymers), methylhydroxyethyl cellulose (MHEC), carboxymethylmethyl cellulose (CMMC), hydrophobically modified carboxymethyl cellulose (HMCMC), hydrophobically modified hydroxyethyl cellulose (HMHEC), hydrophobically modified hydroxypropyl cellulose (HMHPC), hydrophobically modified ethylhydroxyethyl cellulose (HMEHEC), hydrophobically modified carboxymethylhydroxyethyl cellulose (HMCMHEC), hydrophobically modified hydroxypropylhydroxyethyl cellulose (HMHPHEC), hydrophobically modified methyl cellulose (HMMC), hydrophobically modified methylhydroxypropyl cellulose (HMMHPC), hydrophobically modified methylhydroxyethyl cellulose (HMMHEC), hydrophobically modified carboxymethylmethyl cellulose (HMCMMC), cationic hydroxyethyl cellulose (cationic HEC) and cationic hydrophobically modified hydroxyethyl cellulose (cationic HMHEC).

If the polysaccharide of use in the present invention is a guar, the guar may be selected from the group consisting of carboxymethyl guar (CM guar), hydroxyethyl guar (HE guar), hydroxypropyl guar (HP guar), carboxymethylhydroxypropyl guar (CMHP guar), cationic guar, hydrophobically modified guar (HM guar), hydrophobically modified carboxymethyl guar (HMCM guar), hydrophobically modified hydroxyethyl guar (HMHE guar), hydrophobically modified hydroxypropyl guar (HMHP guar), cationic hydrophobically modified hydroxypropyl guar (cationic HMHP guar), hydrophobically modified carboxymethylhydroxypropyl guar (HMCMHP guar) and hydrophobically modified cationic guar (HM cationic guar).

A preferred group of polysaccharides of use in the present invention may be selected from the group consisting of carrageenan, xanthan, guar, PES, starch, starch derivatives, pectin, and mixtures thereof.

In the oral care and household cleaner system of the present invention the acrylic acid polymer may be either a homopolymer or a copolymer.

In the oral care household cleaner system of the present invention the upper limit of the amount of the water soluble/water swellable polymer based on the total weight of the FPS is about 70 wt % more preferably about 50 wt %, more preferably about 40 wt %. Also in the oral care system of the present invention, the lower limit of the amount of the water soluble/water swellable polymer based on the total weight of the FPS is about 10 wt %, more preferably about 20 wt %, still more preferably about 30 wt %.

In accordance with the present invention, the dentrifrice formulations or household cleaner can either be prepared in a continuous or batch process and either in a stepwise addition of the ingredients or a simple dumping of all of the ingredients at once. The order of addition of the ingredients can also vary over a wide range of additions. For example, the functional ingredients can be individually added one at a time to the FPS or all at once or the FPS can be added directly to the formulated ingredients in a single step. Hence, the process of thickening a dentrifrice formulations household cleaners includes adding and mixing a sufficient amount of a stable fluid polymer suspension that is compatible with the dentrifrice formulations household cleaner to thicken the dentrifrice formulations, wherein the stable fluid polymer suspension comprises a water soluble/water swellable polymer, a polyol, and hydrated thickening silica. The resulting functional system has comparable or better rheology and viscosity properties as compared to when using similar thickening agents in dry, solid form. This process is also more efficient and more versatile so that it can be prepared where it can be customized to suit the environment in which it is to operate. This process is extremely user friendly.

The rheology modifiers of the present invention may also be used in combination with other known rheology modifiers including, but not limited to, polysaccharides (e.g., carrageenan, hyaluronic acid, glucosaminoglycan), biopolymers (e.g., xanthan gum), synthetic polymers, and abrasive/thickening silicas.

All percentages and parts are by weight unless specifically stated otherwise.

It should be understood that many variations and modifications are possible without departing from the scope and spirit of this invention.

### EXAMPLES

### Xanthan Gum FPS Preparation

| | |
|---|---|
| 292.5 g | Polyethylene Glycol 400 (PEG 400, Carbowax ® 400 - Dow |
| | Chemicals) |
| 7.5 g | Silica (Aerosil ® 200 from Degussa) |
| 200.0 g | Xanthan Gum (Keltrol ® XG - CPKelco) |

Add Silica to PEG 400. Mix for 1 hour and then add xanthan gum. Mix for one hour.
Stability after 6 weeks: Brookfield® LVT viscosity at 30 rpm, 1100 cps at room temperature. 7% synresis but easy to remix. No hard settling
Stability after 12 weeks: Brookfield® LVT viscosity at 30 rpm, 1090 cps at room temperature. 12% synresis but easy to remix. No hard settling
Stability after 22 weeks: Brookfield® LVT viscosity at 30 rpm, 1030 cps at room temperature. 7% synresis but easy to remix. No hard settling

### Dispersion Properties with Haake Viscometer Test

The xanthan gum FPS (of Example 1 was studied for its dispersion (lumping/no lumping), hydration and dissolution properties with a Haake Model VT501 viscometer. The test was run in deionized water at 25° C., 300 rpm using FL10 sensor. As a comparative example, dry xanthan gum was used

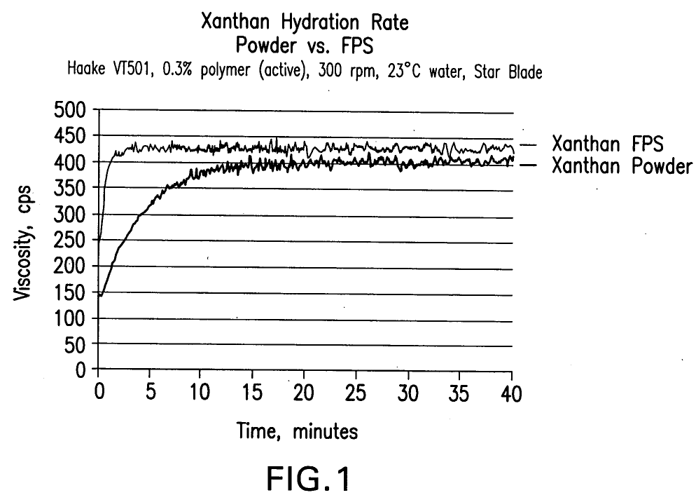

The dispersions were made of examples of the FPS of use in the present invention as well as the comparative examples at 0.3% active xanthan gum concentration Table 1. The dispersions were mixed either for one (1) hour and viscosity was measured as a function of time, FIG. 1. The plots provide viscosity with time as measured by Haake viscometer.

### Comparative Example 1

Powder Xanthan Gum: The Comparative Example 1 took over 60 minutes to reach 100% of its final viscosity. In addition, lumping was observed even after one hour of mixing.

TABLE 1
Hydration Curves Measured with a Haake Viscometer for Example 1
and Comparative Examples 1

| | Concentration in | | | |
|---|---|---|---|---|
| | DI water, by | Time | | |
| Sample | weight (%) | (hrs.) | FIG. | Comments |
| Example. 1 | 0.30 | 1 | 1 | No lumping. Full |
| | | | | viscosity development in |
| | | | | about 3 minutes |
| Comp. Ex. 1 | 0.30 | 1 | 1 | Lumping. No full |
| | | | | viscosity development |
| | | | | after at 60 minutes |

### EXAMPLE 1

Toothpaste Preparation with FPS:
Part 1

| | |
|---|---|
| 13.00 g | Glycerine |
| 16.86 g | Sorbitol |
| 14.26 g | Deionized Water |

Part II

| | |
|---|---|
| 45.00 g | Dicalcium phosphate, Dihydrate (DiCal) |

Part III

| | |
|---|---|
| 1.20 g | Xanthan gum FPS of Example 1 |

Part IV

| | |
|---|---|
| 00.42 g | Tetrasodium pyrophosphate |
| 00.20 g | Saccharin |
| 00.76 g | Sodium monoflurophospate |
| 00.50 g | Sodium Benzoate |
| 06.25 | Deionized water |

Part V

| | |
|---|---|
| 00.55 g | Flavor |
| 01.00 | Sodium Lauryl Sulfate (100% active) |

DiCal of Part II was added to one quart Ross planatory mixer. Next, Part I ingredients were added to the Ross Planatory Mixer. vMix on speed 30 for 10 minutes to wet out Dical. Opened the Ross mixer. Added xanthan gum FPS of Part III and then close the mixer and mix on speed 60 rpm without vacuum. In a separate beaker container combined ingredients of Part IV and heated to about 60° C. to dissolve. Once one hour mixing was completed in Ross mixer for Part I, II, and III, solution of Part IV was added and on speed 45 for 5 minutes. Next vaccum was applied and mixed on speed 60 for 10 minutes. Next, opened the mixer and added ingredients of Part V and mixed for 10 minutes at speed on 30 without vacuum. Open the mixer and scraped down agitators and mixing bowl sides. Closed the mixer and applied vacuum. Mixed at speed 45 for 15 minutes. Reduced mixer speed, shut down mixer and broke vacuum and then toothpaste packed-out in tube. No sign of lumping or gels was observed in final toothpaste.

### Comparative Example 2

Toothpaste Preparation:
Part 1

| 13.00 g | Glycerine |
|---|---|
| 16.86 g | Sorbitol |
| 14.26 g | Deionized Water |
| 01.80 | Polyethylene glycol 400 |

Part II

| | |
|---|---|
| 45.00 g | Dicalcium phosphate, Dihydrate (DiCal) |

Part III

| | |
|---|---|
| 1.20 g | Xanthan gum |

Part IV

| | |
|---|---|
| 00.42 g | Tetrasodium pyrophosphate |
| 00.20 g | Saccharin |
| 00.76 g | Sodium monoflurophospate |
| 00.50 g | Sodium Benzoate |
| 06.25 | Deionized water |

Part V

| | |
|---|---|
| 00.55 g | Flavor |
| 01.00 | Sodium Lauryl Sulfate (100% active) |

DiCal of Part II was added to one quart Ross planatory mixer. Next, Part I ingredients were added to the Ross Planatory Mixer. Mix on speed 30 for 10 minutes to wet out Dical. Opened the Ross mixer. Added xanthan gum of Part III and than close the mixer and mix on speed 60 for one hour without vacuum.

In a separate beaker container combined ingredients of Part IV and heated to about 60° C. to dissolve. Once one hour mixing was completed in Ross mixer for Part I, II and III, solution of Part IV was added and on speed 45 for 5 minutes. Next vacuum was applied and mixed on speed 60 for 10 minutes. Next, opened the mixer and added ingredients of Part V and mixed for 10 minutes at speed on 30 without vacuum. Open the mixer and scraped down agitators and mixing bowl sides. Closed the mixer and applied vacuum. Mixed at speed 45 for 15 minutes. Reduced mixer speed, shut down mixer and broke vacuum and then toothpaste packed-out in tube. Small Gels in final toothpaste.

TABLE 2
Toothpaste for Example 2 and Comparative Examples 2

| | | One day | One Month | |
|---|---|---|---|---|
| | | Viscosity | Viscosity | |
| | Xanthan Gum | Brookfield | Brookfield | |
| | Concentration in | RVT, | RVT, | |
| Sample | toothpaste (%) | 5 rpm cps | 5 rpm cps | Comments |
| Example. 1 | 1.2. | 94,000 | 95,000 | No Gels |
| | | | | or lumping |
| | | | | in toothpaste |
| Comp. Ex. 1 | 1.2 | 96,000 | 97,000 | Small Gels |
| | | | | in toothpaste |

### EXAMPLE 2

### Hard Surface Cleaners

### Formulation

| | |
|---|---|
| 163.6 g | Deionized water |
| 5.0 g | C9-11 alcohol ethoxylate (Neodol ® 91-8) |
| 30.00 g | Citric acid |
| 3.5 g | Product of this invention (40% active suspension of xanthan |
| | gum) |

In beaker, add alcohol ethoxylate to water under agitation. Next add Citric acid and mix until dissolved. Next add Xanthan FPS, the product of this invention. Mix for one hour. No lumps were observed at the end of one hour. Final Brookfield viscosity at 30 rpm 1723 cps at 25° C., pH 1.92.

As a control 1.4 gram of dry commercially available xanthan gum was added to the above formulation in place of product of this invention. Visible lumps were observed at the end of one month. Final Brookfield viscosity at 30 rpm 1568 cps at 25° C., pH 1.99.

What is claimed:

1. An oral care or household cleaner system comprising an abrasive, a preservative, water, detergent and a fluid polymer suspension (FPS) thickening agent comprising
i) a water soluble/water swellable polymer,
ii) a polyol, and
iii) hydrated thickening silica
wherein the water soluble/water swellable polymer is selected from the group consisting of, acrylic acid polymer, salts of acrylic acid polymer, acrylate, acrylamide polymer, polyvinyl pyrolidene, polyvinyl alcohol, clay and polysaccharide, excluding CMC.

2. The oral care or household cleaner system of claim 1 wherein the polysaccharide is a naturally occurring polysaccharide.

3. The oral care or household cleaner system of claim 1 wherein the polysaccharide is a derivatitized polysaccharide.

4 The oral care or household cleaner system of claim 1 wherein polysaccharide is at least one member selected from the group consisting of, guar, locust bean gum, psyllium, gum arabic, gum ghatti, gum karaya, gum tragacanth, gum cassia, fenugreek gum, carrageenan, konjac, agar, alginates, xanthan, scleroglucan, dextran, pectin, starch, chitin, chitosan, processed euchemia seaweed (PES), derivatives and mixtures thereof cellulose ethers (excluding CMC alone).

5. The oral care or household cleaners system of claim 1 wherein the polysaccharide is at least one cellulose ether selected from the group consisting of hydroxyethyl cellulose (HEC), hydroxypropyl cellulose (HPC), water soluble ethylhydroxyethyl cellulose (EHEC), carboxymethylhydroxyethyl cellulose (CMHEC), hydroxypropylhydroxyethyl cellulose (HPHEC), methyl cellulose (MC), methylhydroxypropyl cellulose (MHPC) (when used in combination with other polymers), methylhydroxyethyl cellulose (MHEC), carboxymethylmethyl cellulose (CMMC), hydrophobically modified carboxymethyl cellulose (HMCMC), hydrophobically modified hydroxyethyl cellulose (HMHEC), hydrophobically modified hydroxypropyl cellulose (HMHPC), hydrophobically modified ethylhydroxyethyl cellulose (HMEHEC), hydrophobically modified carboxymethylhydroxyethyl cellulose (HMCMHEC), hydrophobically modified hydroxypropylhydroxyethyl cellulose (HMHPHEC), hydrophobically modified methyl cellulose (HMMC), hydrophobically modified methylhydroxypropyl cellulose (HMMHPC), hydrophobically modified methylhydroxyethyl cellulose (HMMHEC), hydrophobically modified carboxymethylmethyl cellulose (HMCMMC), cationic hydroxyethyl cellulose (cationic HEC) and cationic hydrophobically modified hydroxyethyl cellulose (cationic HMHEC).

6. The oral care or household cleaner system of claim 1 wherein the polysaccharide is at least one guar derivative selected from the group consisting of carboxymethyl guar (CM guar), hydroxyethyl guar (HE guar), hydroxypropyl guar (HP guar), carboxymethylhydroxypropyl guar (CMHP guar), cationic guar, hydrophobically modified guar (HM guar), hydrophobically modified carboxymethyl guar (HMCM guar), hydrophobically modified hydroxyethyl guar (HMHE guar), hydrophobically modified hydroxypropyl guar (HMHP guar), cationic hydrophobically modified hydroxypropyl guar (cationic HMHP guar), hydrophobically modified carboxymethylhydroxypropyl guar (HMCMHP guar) and hydrophobically modified cationic guar (HM cationic guar).

7. The oral care or household cleaner system of claim 1 wherein the polysaccharide is selected from the group consisting of carrageenan, xanthan, guar, PES, starch, starch derivatives, pectin, and mixtures thereof.

8. The oral care or household cleaner system of claim 1 wherein the acrylic acid polymer is a homopolymer or a copolymer.

9. The oral care or household cleaner system of claim 1, wherein the upper limit of the amount of the water soluble/water swellable polymer based on the total weight of the FPS is about 70 wt %.

10. The oral care or household cleaner system of claim 1, wherein the upper limit of the amount of the water soluble/water swellable polymer based on the total weight of the FPS is about 60 wt %.

11. The oral care or household cleaner system of claim 1, wherein the upper limit of the amount of the polysaccharide based on the total weight of the FPS is about 50 wt %.

12. The oral care or household cleaner system of claim 1, wherein the upper limit of the amount of the polysaccharide based on the total weight of the FPS is about 40 wt %.

13. The oral care or household cleaner system of claim 1, wherein the lower limit of the amount of the polysaccharide based on the total weight of the FPS is about 10 wt %.

14. The oral care or household cleaner system of claim 1, wherein the lower limit of the amount of the polysaccharide based on the total weight of the FPS is about 20 wt %.

15. The oral care or household cleaner system of claim 1, wherein the lower limit of the amount of the polysaccharide based on the total weight of the FPS is about 30 wt %.

16. The oral care or household cleaner system of claim 1 wherein the polyol is selected from the group consisting of glycerol, polyethylene glycol, propylene glycol, and mixtures thereof.

17. The oral care or household cleaner system of claim 16 wherein the polyol is polyethylene glycol.

18. The oral care or household cleaner system of claim 1, wherein the upper limit of the amount of the polyol based on the total weight of the FPS is about 90 wt %.

19. The oral care or household cleaner system of claim 1, wherein the upper limit of the amount of the polyol based on the total weight of the FPS is about 80 wt %.

20. The oral care or household cleaner system of claim 1, wherein the upper limit of the amount of the polyol based on the total weight of the FPS is about 70 wt %.

21. The oral care or household cleaner system of claim 1, wherein the lower limit of the amount of the polyol based on the total weight of the FPS is about 40 wt %.

22. The oral care or household cleaner system of claim 1, wherein the lower limit of the amount of the polyol based on the total weight of the FPS is about 30 wt %.

23. The oral care or household cleaner system of claim 1, wherein the lower limit of the amount of the polyol based on the total weight of the FPS is about 50 wt %.

24. The oral care or household cleaner system of claim 1, wherein the lower limit of the amount of the polyol based on the total weight of the FPS is about 60 wt %.

25. The oral care or household cleaner system of claim 1, wherein the upper limit of the amount of the hydrated thickening silica based on the total weight of the FPS is about 5.0 wt %.

26. The oral care or household cleaner system of claim 1, wherein the upper limit of the amount of the hydrated thickening silica based on the total weight of the FPS is about 2.0 wt %.

27. The oral care or household cleaner system of claim 1, wherein the lower limit of the amount of the hydrated thickening silica based on the total weight of the FPS is about 0.1 wt %.

28. The oral care or household cleaner system of claim 1, wherein the lower limit of the amount of the hydrated thickening silica based on the total weight of the FPS is 0.5 wt %.

29. The oral care or household cleaner system of claim 4, wherein the polysaccharide is xanthan gum.

30. The oral care or household cleaner system of claim 1, wherein the hydrated thickening silica is a fumed silica that has a small particle size of about 10 μm and a large surface area in the range of from about 200 to 300 square meters per gram.

31. The oral care or household cleaner system of claim 29, wherein the hydrated thicker silica is a fumed silica, and wherein the polyol is polyethylene glycol.

32. A process of thickening an oral care or household cleaner system of any of the preceding claims, comprising adding a sufficient amount of a fluid polymer suspension to an oral care or household cleaner system wherein the fluid polymer suspension comprises a water soluble/water swellable polymer, a polyol, and hydrated thickening silica.
